(19) United States
(12) Patent Application Publication
(10) Pub. No.: US 2022/0386591 A1
(43) Pub. Date: Dec. 8, 2022

(54) CRYOPRESERVATION COMPOSITIONS AND METHODS INCLUDING GLYCEROL ESTER COMPONENTS

(71) Applicant: THE SECANT GROUP LLC, Telford, PA (US)

(72) Inventors: Brian GINN, Harleysville, PA (US); Stephanie REED, Conshohocken, PA (US); Peter D. GABRIELE, Frisco, TX (US); Jeremy J. HARRIS, Doylestown, PA (US); Scott RADZINSKI, Gilbertsville, PA (US)

(21) Appl. No.: 17/805,100

(22) Filed: Jun. 2, 2022

Related U.S. Application Data
(60) Provisional application No. 63/196,738, filed on Jun. 4, 2021.

(51) Int. Cl. A01N 1/02; C12N 5/00

(52) U.S. Cl. CPC A01N 1/0221; C12N 5/0018; C12N 5/0068; A01N 1/0284

(57) ABSTRACT

A cryopreservation process includes combining a cryopreservation composition with a biological sample. The cryopreservation composition includes at least one glycerol ester component. The cryopreservation process also includes then cooling the cryopreservation composition with the biological sample to a cryopreservation temperature. The cryopreservation composition aids in cryopreserving the biological sample at the cryopreservation temperature. A cryopreservation composition includes at least one glycerol ester component. A cryopreserved system includes a biological sample in a cryopreservation composition at a cryopreservation temperature. The cryopreservation composition includes at least one glycerol ester component.

### CROSS-REFERENCE TO RELATED APPLICATIONS

This application claims priority to and the benefit of U.S. Provisional Application No. 63/196,738 filed Jun. 4, 2021, which is hereby incorporated by reference in its entirety.

### FIELD OF THE INVENTION

The present disclosure is generally directed to compositions and methods for the cryopreservation of biological materials susceptible to damage under cryopreservation conditions. More specifically, the present disclosure is directed to compositions and methods including a glycerol ester component for cryopreservation of proteins, cells, tissues, or organs.

### BACKGROUND OF THE INVENTION

Proteins are ubiquitous in the biotechnology, bioprocessing, and biopharmaceutical industries, often serving as therapeutics, reagents, biocatalysts, and/or food supplements. Many proteins have a limited storage lifetime, leading to the frequent use of freezing during handling and storage to increase their storage time. Proteins may degrade, become physically and/or chemically unstable, become inactivated, and/or irreversibly aggregate under environmental stresses, such as temperature, sunlight, hydration, and dehydration. One conventional method of protein freezing is lyophilization of a protein therapeutic agent that is pre-filled into a syringe for later reconstitution into an injectable suspension for in vivo use.

Cells are increasingly utilized in biotechnology, bioprocessing, and biopharmaceutical industries as well, commonly serving, for example, as therapeutics, as vehicles for therapeutics, as factories for therapeutics, as factories for biofuels, as factories for biocatalysts, in methods for fertility, and in donor tissues. Similar to proteins, cells are conventionally frozen during storage and shipping to maintain viability. One example is controlled freezing of an apheresis product for extended cryostorage prior to delivery to a processing facility. Furthermore, cryopreservation of the finished therapeutic product is often used upon return to the treatment facility.

Preventing ice recrystallization and inhibiting ice growth during cryostorage protects tissues, cells, proteins, and other intra-cellular components against freeze-stress. Common conventional cryoprotectants include organic solvents, such as, for example, glycerol, trehalose, and dimethyl sulfoxide (DMSO). Since DMSO generally permeates well through the cell membrane and into the cytoplasm, it helps prevent intracellular, as well as extracellular, ice crystal formation during freezing. Conventional cryoprotectants, however, may harm, alter, and/or be toxic to cells. Conventional cryopreservation additives also may interfere with various bioproces sing techniques, surface materials, and analyses.

Conventional methods to cryoprotect proteins include using covalent polymer-protein conjugates to freeze proteins, but this typically is complex, reduces protein function, and generates a new molecular species, which must be tested for safety and efficacy. To combat this, osmolytes may be added in large concentrations prior to lyophilization, spray drying, vacuum foam drying, or direct freezing, but there are biocompatibility concerns with osmolytes being present post-thaw.

As an alternative to these conventional approaches, polymers may be used to inhibit ice recrystallization. For example, polymers, such as polyethylene glycol (PEG) and polyvinyl alcohol (PVA) of different molecular weights, that mimic antifreeze proteins may be used to disrupt and inhibit ice crystal growth. Irreversible protein aggregation due to ice crystal growth is a major cause of cryo-damage, and when prevented, allows proteins to retain activity post-thaw.

### BRIEF DESCRIPTION OF THE INVENTION

There is a need for cryopreservation compositions that are biocompatible while still protecting the function and/or viability of proteins, cells, tissues, or organs under cryopreservation conditions and during freezing and thawing.

In exemplary embodiments, a cryopreservation process includes combining a cryopreservation composition with a biological sample. The cryopreservation composition includes at least one glycerol ester component. The cryopreservation process also includes then cooling the cryopreservation composition with the biological sample to a cryopreservation temperature. The cryopreservation composition aids in cryopreserving the biological sample at the cryopreservation temperature.

In exemplary embodiments, a cryopreservation composition includes at least one glycerol ester component.

In exemplary embodiments, a cryopreserved system includes a biological sample in a cryopreservation composition at a cryopreservation temperature. The cryopreservation composition includes at least one glycerol ester component.

### BRIEF DESCRIPTION OF THE DRAWINGS

FIG. 1 shows pre-freeze and post-thaw viability of cells in various media, where the cells were frozen immediately after addition of the cryopreservative.

FIG. 2 shows pre-freeze and post-thaw viability of cells in various media, where the cells were incubated for four hours after addition of the cryopreservative prior to freezing.

Wherever possible, the same reference numbers will be used throughout the drawings to represent the same parts.

### DETAILED DESCRIPTION OF THE INVENTION

Provided are compositions and methods for cryopreservation including glycerol ester components.

Embodiments of the present disclosure, for example, in comparison to concepts failing to include one or more of the features disclosed herein, provide a safe, non-cytotoxic, nutrient-rich media additive as a metabolic cryoprotectant during bioprocessing, including frozen protein storage or cell maintenance, cell expansion, or frozen cell storage; provide protection, stabilization, or nutrients to cells before, during, or after cryostorage; provide cryoprotection that is non-fouling, non-interfering, biodegradable, or metabolizable by cells, to expedite, facilitate, or improve downstream processing; or combinations thereof.

In exemplary embodiments, a cryopreservation composition includes at least one glycerol ester component. The cryopreservation composition preferably also includes a medium selected to support a target biological sample at a physiological temperature prior to and after cryopreservation of the biological sample. In exemplary embodiments, additional cryopreservation components that are specifically advantageous to the target biological sample are selected and provided in the cryopreservation composition.

In exemplary embodiments, the cryopreservation composition is free of DMSO.

The glycerol ester component is provided in the cryopreservation composition in a form and in an amount sufficient for cryopreservation of a target biological sample. In conventional cryopreservation compositions, DMSO may be present at about 10% v/v in an aqueous composition. In exemplary embodiments, the glycerol ester component is present in the cryopreservation composition in an amount, by weight, of about 10% or less, alternatively about 0.01% to about 10%, alternatively about 0.1% to about 10%, alternatively about 1% to about 10%, alternatively about 9% or less, alternatively about 8% or less, alternatively about 7% or less, alternatively about 6% or less, alternatively about 5% or less, alternatively about 1% to about 5%, alternatively about 4% or less, alternatively about 3% or less, alternatively about 2% or less, alternatively about 1% or less, alternatively about 1% to about 0.01%, alternatively about 0.1% or less, alternatively about 0.1% to about 0.01%, or any value, range, or sub-range therebetween.

In some embodiments, cryopreservation composition includes the glycerol ester component in an aqueous solution. In some embodiments, the aqueous solution is a cell media, such as, for example, a cell culture media.

The solubility of a glycerol ester component in water may decrease with increasing molecular weight, limiting its concentration at higher molecular weights in a cryopreservation composition. In some embodiments, the form of the glycerol ester component includes a functionalization that increases the water solubility of the functionalized glycerol sebacate component to allow it to be included in greater amounts in a cryopreservation composition.

In some embodiments, the cryopreservation composition contains one or more additives. Appropriate additives include, but are not limited to, pH buffering agents or stabilizers. Appropriate pH buffering agents include, but are not limited to, sodium phosphate or citrate buffers. Appropriate stabilizers include, but are not limited to, sugars, such as, for example, glucose, fructose, sucrose, or lactose.

In some embodiments, the glycerol ester component inhibits ice crystal formation in a cryopreservation system to aid in cryopreserving a biological sample. In some embodiments, the biological sample includes cells. In yet other embodiments, the biological sample includes proteins. In other embodiments, the biological sample includes nucleic acids. The glycerol ester component may provide a similar inhibitory effect of inhibiting ice crystal formation to benefit the cells, proteins, or nucleic acids being cryopreserved. When the biological sample is cells, a low molecular weight portion of the glycerol ester component may more easily enter cells than higher molecular weight portions to reduce intracellular ice crystal formation, thereby improving cryopreservation of the cells.

In exemplary embodiments, the cryopreservation of a biological sample includes combining a cryopreservation composition with the biological sample and then cooling the biological sample to a cryopreservation temperature. The cryopreservation composition includes at least one glycerol ester component. The biological sample may include cells, tissue, organ, and/or proteins.

In some embodiments, the combining includes adding the glycerol ester component to media containing the biological sample. In some embodiments, the combining occurs at a physiological temperature.

In some embodiments, the cryopreservation includes incubating the biological sample in the cryopreservation composition for a predetermined incubation period of time after the combining and prior to the cooling. In some embodiments, the incubation occurs at a physiological temperature. An appropriate incubation period of time is in the range of about one hour to about eight hours, alternatively about one hour to about four hours, alternatively about two hours to about eight hours, alternatively about two hours to about six hours, alternatively about three hours to about five hours, alternatively about four hours, or any value, range, or sub-range therebetween.

In exemplary embodiments, the cooling includes decreasing the temperature of the biological sample from a physiological temperature to a cryopreservation temperature. Some biological samples cryopreserve better with a rapid cooling, whereas other biological samples cryopreserve better with a slow cooling. In exemplary embodiments, the temperature decrease profile is selected based on the behavior of the target biological sample that is being cryopreserved. In exemplary embodiments, the temperature decrease occurs over a period of a few hours for cryopreservation of cells, while the temperature decrease may occur rapidly within minutes for cryopreservation of proteins.

In exemplary embodiments, the biological sample is heated from the cryopreservation temperature after a predetermined period of time, and preferably returned to a physiological temperature with all or almost all of the biological activity of the biological sample being restored. As with freeze-down, some biological samples are restored better with a rapid temperature increase, whereas other biological samples are restored better with a slow temperature increase. In exemplary embodiments, the temperature increase profile is selected based on the behavior of the target biological sample that has been cryopreserved. In exemplary embodiments, the temperature increase occurs rapidly over a period of minutes for thawing of cells, while the temperature increase may occur slowly over a period of up to a few hours for thawing proteins, depending on the structure of the protein.

In exemplary embodiments, the cryopreservation composition aids in cryopreserving the biological sample at the cryopreservation temperature. When the biological sample includes cells, the cryopreservation composition aiding in cryopreserving the cells increases the percentage of viable cells post-thaw compared to cryopreserving using a composition lacking the at least one glycerol ester component. When the biological sample includes proteins, the cryopreservation composition aiding in cryopreserving the protein increases the level of protein activity post-thaw compared to cryopreserving using a composition lacking the at least one glycerol ester component.

As used herein, cryopreservation temperature refers to a temperature of −20° C. or below. In exemplary embodiments, the cryopreservation temperature is in the range of about −20° C. to about −200° C., alternatively about −20° C. to about −80° C., alternatively about −20° C. to about −140° C., alternatively about −80° C. or below, alternatively about −80° C. to about −200° C., alternatively about −80° C. to about −140° C., alternatively about −140° C. or below, alternatively about −140° C. to about −200° C., alternatively about −190° C. to about −200° C., alternatively about −80° C., alternatively about −140° C., alternatively about −196° C., or any value, range, or sub-range therebetween.

As used herein, cryopreservation conditions refers to storage at a cryoprotection temperature for a period of time of one day or longer. Cryopreservation conditions may also include an osmotic condition or a pH condition that may be outside a normal physiological range but may benefit the cryopreservation at the cryopreservation temperature.

As used herein, a physiological temperature refers to a temperature of about 20° C. or greater at which the biological sample is active or viable. In exemplary embodiments, the physiological temperature is in the range of about 20° C. to about 40° C., alternatively about 20° C. to about 30° C., alternatively about 30° C. to about 40° C., alternatively about 35° C. to about 40° C., alternatively about 37° C., or any value, range, or sub-range therebetween.

As used herein, glycerol ester component refers to any component having at least one repeat unit of glycerol and a diacid coupled by an ester bond.

In exemplary embodiments, the diacid has the formula [HOOC(CH₂)ₙCOOH], where n=1-30. Appropriate diacids may include, but are not limited to, malonic acid, succinic acid, glutaric acid, adipic acid, pimelic acid, suberic acid, azelaic acid, or sebacic acid. In some embodiments, the diacid is sebacic acid. In some embodiments, the glycerol ester component also includes one or more other acids, such as, for example, monoacid small chain fatty acids.

In some embodiments, the glycerol ester component is a co-oligomer of glycerol and a diacid.

In some embodiments, the glycerol ester component is a copolymer of glycerol and a diacid.

In exemplary embodiments, the glycerol ester component is a glycerol-sebacate component having at least one glycerol-sebacate repeat unit.

In exemplary embodiments, the glycerol-sebacate component consists of or consists essentially of glycerol and sebacate units.

In exemplary embodiments, the glycerol ester component has repeating units of (glycerol sebacate). In some embodiments, the glycerol ester component is in the form of poly(glycerol sebacate). In some embodiments, the glycerol ester component is in the form of oligomeric (glycerol sebacate) (OGS).

Poly(glycerol sebacate) is a crosslinkable elastomer formed as a co-polymer from glycerol and sebacic acid. Poly(glycerol sebacate) is biocompatible and biodegradable, reduces inflammation, improves healing, and has antimicrobial properties, all of which make it useful as a biomaterial.

As used herein, PGS refers to a polymer of (glycerol sebacate) having a weight average molecular weight of greater than 10,000.

As used herein, OGS refers to an oligomer of (glycerol sebacate) having a weight average molecular weight of 10,000 or less.

In some embodiments, the glycerol ester component is made by a method disclosed in U.S. Pat. No. 9,359,472, issued on Jun. 7, 2016, and entitled “Water-Mediated Preparations of Polymeric Materials”, which is hereby incorporated by reference, such as, for example, to provide a glycerol ester component having a low polydispersity index.

In exemplary embodiments, the glycerol ester component includes molecular weights in the low molecular weight range of 200 Da to 1200 Da, alternatively 300 Da to 1200 Da, or any value, range, or sub-range therebetween, to remain water soluble and penetrate cells. The preferred low molecular weight range may depend on the molecular weight of the acid component of the glycerol ester.

In exemplary embodiments, the glycerol ester component is customized for cryopreservation of the biological sample. The customization may include selection of a stoichiometric ratio of the glycerol to the diacid, a molecular weight, and/or a polydispersity index for the glycerol ester component. Appropriate values for the stoichiometric ratio of glycerol:diacid may include between 1:0.25 and 1:2, alternatively between 1:0.5 and 1:1.5, alternatively between 1:0.75 and 1:1.25, or any value, range, or sub-range therebetween. Appropriate values for the polydispersity index for the glycerol ester component may include about 7.5 or less, alternatively about 7 or less, alternatively about 6.5 or less, or any value, range, or sub-range therebetween.

In some embodiments, the cryopreservation composition includes chemical functional moieties in addition to those provided by glycerol and diacid. The additional functional moieties may be included separately from the glycerol ester component or attached covalently to the backbone or to an end group of the glycerol ester component as a modification of the glycerol ester component. In some embodiments, chemical functional moieties are provided by covalent attachment of one or more modifications to the glycerol ester component. Appropriate modifications may include, but are not limited to, a urethane, an acrylate, an amino acid, a peptide, a fatty acid, a diacid smaller than sebacic acid, a multifunctional acid, a sugar, a cholesterol, or a vitamin.

In some embodiments, the glycerol ester component includes one or more additional crosslinking chemistries, such as photochemistry crosslinking or such as provided by the presence of urethane or acrylate. In some embodiments, the glycerol ester component includes poly(glycerol sebacate) urethane (PGSU). In some embodiments, the glycerol ester component includes poly(glycerol sebacate) acrylate (PGSA).

In some embodiments, the cryopreservation composition includes hydrogel formulations including a glycerol ester component. In some embodiments, the glycerol ester component includes one or more hydrogel branches attached covalently to the backbone or to an end group of the glycerol ester component. Such hydrogel formulations may also include PEG.

In some embodiments, the cryopreservation composition includes formulations with amino acids, peptides, and/or fatty acids, which may be included separately from the glycerol ester component or attached covalently to the backbone or to an end group of the glycerol ester component, both for function and for limiting molecular weight growth during synthesis.

In some embodiments, the cryopreservation composition includes smaller diacids or multifunctional acids, such as, for example, citric acid, which may be included separately from the glycerol ester component or attached covalently to the backbone or to an end group of the glycerol ester component.

In some embodiments, the cryopreservation composition includes a glycerol ester component in the form of a ligand-conjugated PGS, where a collection of entities are held together through ligand coordination that then decouple once in the media.

In some embodiments, the cryopreservation composition includes small sugars, cholesterols, and/or vitamins, such as, for example, vitamin D (calciferols). These components may be included separately from the glycerol ester component or attached covalently to the backbone or to an end group of the glycerol ester component, to improve solubility and cell membrane transport.

In some embodiments, the cryopreservation composition includes PGS that has been three-dimensionally formed to mimic the topology and structure of an antifreeze protein.

In some embodiments, the structure of the glycerol ester component is selected to penetrate cells to intra-cellularly protect them from freeze stress.

In some embodiments, the structure of the glycerol ester component is selected to have an affinity for the cellular membrane to stabilize it during freezing. For example, the relative amounts of glycerol and sebacic acid in a glycerol-sebacate component may be selected such that the glycerol-sebacate component interacts with the cell membrane either through lipophilic, electrostatic, or hydrogen bonding interaction to plasticize the cell membrane. In other embodiments, the glycerol ester component is modified with one or more moieties to promote the desired interaction with the cell membrane. Such appropriate moieties may include, but are not limited to, cationic moieties, anionic moieties, or lipid moieties and preferably maintain the non-immunogenicity of the glycerol ester component.

Appropriate cationic moieties may include, but are not limited to, amines, ammonium, amino acids, peptides, peptide sequences such as the cell adhesion promoting arginine-glycine-aspartic acid (RGD), choline, phosphocholine, sodium ions, potassium ions, or calcium ions.

Appropriate anionic moieties may include, but are not limited to, sulfates, phosphates, sulfonates, sulfites, carboxy salts, carbohydrates, or glycoproteins.

Appropriate lipid moieties may include, but are not limited to, single-tail lipids, double-tail lipids, or phospholipids, which may be PEGylated.

In some embodiments, the structure of the glycerol ester component is selected to remain outside cells to extra-cellularly stabilize them and the surrounding environment during freezing.

In some embodiments, the structure of the glycerol ester component is selected such that the glycerol ester component penetrates the cell membrane and becomes present in the cytosol or cytoplasm of the cell.

In some embodiments, the structure of the glycerol ester component is selected to depress the freezing temperature, either intra-cellularly, extra-cellularly, or both.

In some embodiments, a mixture of two or more different glycerol ester components are used to specifically accumulate the glycerol ester component in two or more of the following components: extracellular space, intercellular space, cell membrane, cell cytoplasm, cell cytosol, cell nucleus, cell organelles.

In some embodiments, a mixture of two or more differing glycerol esters having differing efficacies of cryopreservation for differing cell types are used.

In some embodiments, a glycerol ester is used that has a selectively better cryoprotective effect on one or more particular cell types over other cell types in a mixture of cells as a method to selectively cryopreserve cells of a desired type and to not cryopreserve cells of an unwanted cell type.

In some embodiments, the cryopreservation composition and cells, in combination, form an emulsion, vesicles, or coacervates that have a cellular rich center with an anti-freeze formulation including the glycerol ester component that protects from exterior ice formation.

In some embodiments, cell culture media is supplemented with the glycerol ester component to form the cryopreservation composition.

In some embodiments, the cells are immediately frozen following exposure to culture media containing the glycerol ester component.

In some embodiments, the cells are exposed to culture media containing the glycerol ester component for a particular duration of time before freezing.

In some embodiments, the glycerol ester component is in the form of one or more PGS microcarriers, such as PGS microspheres or a PGS coating of a textile construct, on which the cells to be cryopreserved are cultured. In some embodiments, the PGS microcarriers are porous. In some embodiments, the PGS microcarriers are hollow.

In some embodiments, a PGS-coated textile is housed inside a biocontainment vessel. In some embodiments, the PGS-coated textile lines the inside surfaces of a biocontainment vessel. In some embodiments, the PGS coating is in the form of a hydrogel that reduces ice crystal formation at the walls of the biocontainment vessel under cryopreservation conditions. Hydrogels are capable of suppressing ice crystallization during cryopreservation by having a tight association of water at the material interface and thereby providing the benefit of reducing ice nucleation sites leading to the suppressed ice crystallization. In some embodiments, the PGS coating contains citric acid as an anticoagulant.

When applied as an interior film to a biocontainment vessel, a PGS coating may redirect or inhibit propagation of ice crystals. Normal freezing of a volumetric container occurs isotropically inward from the outside, and crystals propagate accordingly from nucleation sites at the solid-liquid interface. A PGS coating may provide a variable spatial distribution of wettability and surface energy based on polar (glycerol ester) and non-polar (sebacic acid esters) surface domains. The PGS-coated solid-liquid interface may retard or dampen nucleation and the freezing organization, acting as a solid-state anti-freeze. The contents of the vessel may still freeze, but the PGS film may mitigate or redirect ice needle formation and ice crystallization propagation. This phenomenon may prevent ice crystals from puncturing cells and reducing cell viability when thawed.

In some embodiments, at least a portion of the glycerol ester component permeates into the cell membrane to intra-cellularly stabilize cells and thereby may serve as a non-toxic metabolic cryopreservative. In some embodiments, at least a portion of the glycerol ester component remains extra-cellular, such as where cryoprotection may be achieved without cell membrane penetration and serves as a non-toxic metabolic cryopreservative. In exemplary embodiments, the glycerol ester component serves as a cell cryoprotectant as well as boosting cell proliferation and metabolic behavior.

Proteins may be prone to irreversibly aggregate, thereby reducing their efficacy. The likelihood of aggregation may depend on freezing rate, starting freezing temperature, ending freezing temperature, presence or lack of presence of stabilizers in the solution used during lyophilization, ionic strength of the solution, and/or the concentration of the protein. These factors may be optimized during downstream processing of a protein during manufacturing.

In exemplary embodiments, a cryopreservation composition includes a glycerol ester component that similarly inhibits ice crystal growth and associated irreversible protein aggregation, thereby serving as a non-toxic cryopreservative that is biocompatible for downstream uses in vivo.

In some embodiments, a cryopreservation process includes freezing the protein in a cryopreservation composition at a temperature of about −30° C. or lower and lyophilizing the composition at a temperature of about −80° C. In some embodiments, the cryopreservation process further includes storing the lyophilized protein at a temperature of about 20° C. or less, such as, for example, at about 4° C. or less, which may prevent microbial growth in the sample.

When the glycerol ester component includes PGS or OGS, the glycerol:sebacic acid ratio may be altered to modify its hydrophilic-hydrophobic balance as well as its charge and polarity to act as a protein cryoprotectant.

In exemplary embodiments, the glycerol:sebacate ratio and the molecular weight are selected based on what best interacts with the protein or proteins to be protected. These properties may serve to ionically complex the glycerol ester component with the protein, creating a complex coacervation, which may help to protect the protein from harsh environments once reconstituted, may help to extend the active lifetime of the protein via circulation life or half-life, and/or may help sustain delivery of the protein in a long-acting drug formulation.

PEG and PVA, which are conventionally used as protein cryoprotectants, do not have the same functional group content as PGS, are not customizable like PGS beyond molecular weight, may not provide similar hydrophilic-hydrophobic functionality or charge and polarity as PGS, and accordingly may not be effective at trapping proteins in a complex coacervation.

In exemplary embodiments, the glycerol ester component includes soluble low molecular weight fractions that may directly replace DMSO and glycerol usage as cryoprotectants. Although a glycerol ester component may have a cryoprotectant effect similar to that of glycerol, the glycerol ester component is effective at much lower concentrations such that less osmotic pressure damage occurs to cells.

In exemplary embodiments, the soluble glycerol ester component has a molecular weight less than about 1200 Da with a linear sebacate region capped with glycols to restrict ice crystal formation. In soluble form, the soluble glycerol ester component cryoprotectant is solubilized in a carrier solution with an osmotic pressure of about 280 milliosmole (mOsm) or greater and then added to the cells requiring cryopreservation. In exemplary embodiments, the osmotic pressure is in the range of about 280 mOsm to about 320 mOsm, but an even higher osmotic pressure may help better drive the soluble glycerol ester component into the cells. Additionally, cells tend to do better under cryopreservation conditions when at higher osmotic pressure rather than lower, since they exude water and shrink to increase their internal osmotic pressure under such conditions. This action reduces the amount of intracellular water, thereby reducing the likelihood and amount of ice crystal formation, and increases the density of the cell membrane, making it more resistant to puncture by ice. Cells may then be frozen to a sub-freezing cryopreservation temperature and stored.

Once frozen, cells are typically stored at cryogenic temperatures (liquid nitrogen liquid phase or vapor phase) to slow down ice crystal growth that occurs even if the solution is already frozen. Such cryogenic temperatures are conventionally about −200° C. In exemplary embodiments, a cryopreservation composition including a glycerol ester component allows cells to be frozen and stored for a longer period of time at a warmer freezing temperature, such as in the range of −80° C. to −20° C., while still preserving cell viability. Cryopreservation may occur, however, at any temperature in the range of −200° C. to −20° C., alternatively −200° C. to −80° C., alternatively −80° C. to −20° C., or any value, range, or sub-range therebetween. When particularly effective at cryopreservation, cryopreservation compositions including a glycerol ester component may enable prolonged storage at higher frozen temperatures, such as in the range of −80° C. to −20° C., at which conventional cryopreservation composition have the issue of ice crystal sizes slowly increasing over time, eventually piercing cells. Upon thawing of the cells, the glycerol ester component may need not be separated from the cells in the thawed cell solution but may remain and act as a metabolic booster to reduce recovery time of the cells from cryopreservation.

In exemplary embodiments, the glycerol ester component includes one or more PGS microcarriers. The PGS microcarrier may be of any form or composition that simplifies, reduces steps, reduces time, and/or reduces the cost associated with cell therapy, cell expansion, bioprocessing, and/or manufacturing. Specifically, when the PGS microcarriers are PGS microspheres, cells may be frozen directly on the PGS microspheres for cryostorage without the need to remove the cells, as a result of the presence of PGS degradation products, extractables, and/or leachables that have released into the media and provide cryoprotection to the cells. Some of these PGS byproducts may penetrate the cells, while others may remain extra-cellular.

In some embodiments, the cryopreservation composition includes PGS microcarriers with anti-ice nucleation polymers grafted to the surface of the PGS microcarriers to inhibit ice crystal formation. In such embodiments, the microcarriers act as an inhibitor of ice crystal progression throughout the solution to enhance the effects by a surface coating. In such embodiments, the cells need not be attached to the PGS microcarriers.

Cells may be thawed directly on the PGS microspheres after cryostorage is complete, and the PGS byproducts that are still present in the surrounding media may provide nutrients that the cells can metabolize immediately. Cells may be injected into the body while still adhered to the PGS microspheres, improving cell viability, homing, function, metabolism, and/or residence duration at the tissue injection site. The end result is a cell microcarrier that may act as a substrate, an antifreeze, a nutrient, and a delivery vehicle simultaneously.

This process offers an end-to-end solution that may simplify the cell therapy manufacturing process. This process may eliminate the extra processing steps and time involved in changing media solutions multiple times. This process may also eliminate the processing steps and time involved in harvesting the cells off the microcarriers and then neutralizing the removal enzyme, such as trypsin. This process may also eliminate any filtration steps involved in separating the cells from the microcarriers. In certain applications, however, the use of PGS microcarriers may introduce alternative additional processing steps over conventional cryopreservation processes.

In some embodiments, a PGS film in solid state form is applied to the interior walls of a cryopreservation storage device, such as, for example, a soft plastic container or a hard plastic cryovial. In such embodiments, the application of PGS to the interior walls may reduce the density of nucleation sites on the interior of the bag, and low molecular weight fractions of PGS may be released from the coating into the solution to reduce ice crystal formation.

In some embodiments, a PGS film in solid state form, which is applied to the interior walls of a cryopreservation storage device, contains dangling pendant molecules of glycerol esters to reduce ice crystal nucleation sites.

A glycerol ester component may be included for any appropriate application, where its ability to reduce or eliminate ice crystallization would be advantageous.

In some embodiments, a glycerol ester component may serve as an adjuvant in lyophilized protein formulations of biopharmaceutical products.

In some embodiments, a glycerol ester component may serve as an adjuvant in lyophilized polymer microsphere formulations, such as poly(lactic-co-glycolic acid) (PLGA)-based microspheres, in drug delivery products.

In some embodiments, a glycerol ester component may serve as a cryoprotectant to prevent irreversible aggregation of nano-particles or micro-particles during lyophilization.

In some embodiments, a glycerol ester component may be used as a stabilizer for polyplexed molecules.

In some embodiments, a glycerol ester component may be used as a stabilizer for lipid-based nanoparticles.

In some embodiments, a glycerol ester component may be used as a stabilizer for cationic lipid particles.

In some embodiments, a glycerol ester component may be used as a stabilizer for liposome particles.

In some embodiments, a glycerol ester component may serve as a cryomedium for tissue embedding, since it may preserve the tissue structure during freezing and also during cryo sectioning.

In some embodiments, a glycerol ester component is included in a cryopreservation system that applies directional freezing to control ice crystal nucleation, formation, growth, and/or directional geometry. This directional freezing may, in turn, control the polymer structure, porosity, and directional geometry of the glycerol ester component. This may result in different cryopreservation properties or different interactions with cells. In addition, during ice crystal growth in directional freezing, particulates are preferentially excluded and partitioned away from the advancing ice crystal, creating some regions that are rich in ice and other regions that are poor in ice. This may permit a method to create sequestered cellular clusters in PGS-dense regions separated by ice crystals that may then remain in place, be thawed to create a particular pattern of living cells in a PGS matrix, or be lyophilized to create pores within a freeze-dried tissue structure.

In some embodiments, a glycerol ester component may serve as a cryoprotectant for freeze-dried foods, preserving the native structure and architecture of the plant-based or meat-based food product.

### EXAMPLES

The invention is further described in the context of the following examples, which are presented by way of illustration, not of limitation.

### Example 1

A glycerol ester component in the form of PGS was added to cell culture media containing Jurkat E6.1 cells (ATCC #TIB-152) at three different concentrations: 250 μg/mL (about 0.025 wt %), 500 μg/mL (about 0.05 wt %), and 1000 μg/mL (about 0.1 wt %). The PGS had a weight average molecular weight of about 20,000. Cell culture media containing Jurkat E6.1 cells (ATCC #TIB-152) with no PGS and cell culture media containing Jurkat E6.1 cells (ATCC #TIB-152) with 10% DMSO served as controls. Cells were then immediately frozen at a rate of −1° C./min to −80° C. Once frozen, the cryopreserved cells were transferred to liquid nitrogen vapor phase storage for 72 hours. Following storage in a liquid nitrogen vapor phase, the cells were thawed and their viability was assessed through a trypan blue exclusion assay using a ThermoFisher Countess II FL automated cell counter to determine the percentage of viable cells.

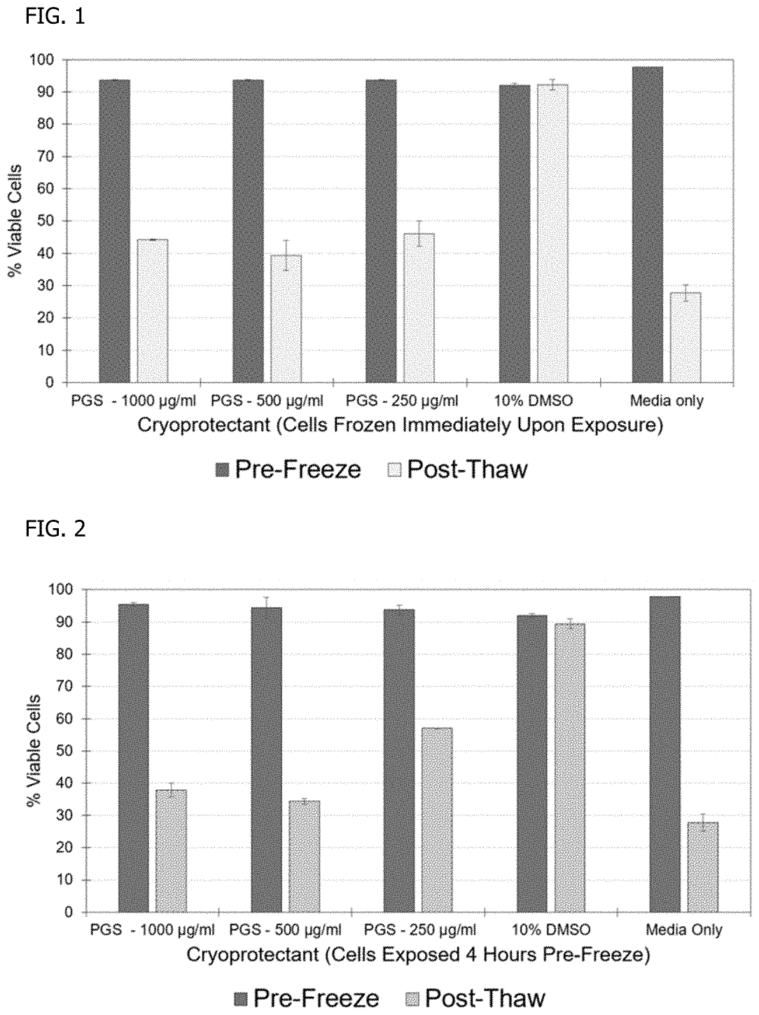

FIG. 1 shows that the addition of PGS as a cryoprotectant improved viability of the Jurkat cells following thawing compared to the use of cell culture media that did not contain any cryopreservation components. With cell culture media, only about 28% of the cells were viable post-thaw. In 250 μg/mL of PGS, about 46% of the cells were viable post-thaw. Increasing the amount of PGS did not further increase cell viability levels. In 500 μg/mL of PGS, about 40% of the cells were viable post-thaw. In 1000 μg/mL of PGS, about 44% of the cells were viable post-thaw. For a positive control, FIG. 1 shows that addition of 10 wt % of DMSO provided cryoprotection to essentially all of the viable cells.

### Example 2

A glycerol ester component in the form of PGS was added to cell culture media containing cells as in Example 1, but the cells were then incubated for four hours after addition of the PGS cryoprotectant and prior to freezing rather than immediately freezing after the addition of PGS. The PGS had a weight average molecular weight of about 20,000. The rest of the process remained the same as in Example 1.

A comparison of FIG. 1 to FIG. 2 shows that the four-hour incubation with PGS improved cell viability relative to immediate freezing for PGS at 250 μg/mL, but cell viability was worse for PGS at 500 μg/mL and 1000 μg/mL with a four-hour incubation with the PGS compared to immediate freezing. For 250 μg/mL of PGS, post-thaw cell viability increased from about 46% to about 57%. For 500 μg/mL of PGS, post-thaw cell viability decreased from about 40% to about 34%. For 1000 μg/mL of PGS, post-thaw cell viability decreased from about 44% to about 38%.

All above-mentioned references are hereby incorporated by reference herein.

While the invention has been described with reference to one or more exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A cryopreservation process comprising:
combining a cryopreservation composition with a biological sample, wherein the cryopreservation composition comprises at least one glycerol ester component; and then
cooling the cryopreservation composition with the biological sample to a cryopreservation temperature, wherein the cryopreservation composition aids in cryopreserving the biological sample at the cryopreservation temperature.

2. The cryopreservation process of claim 1 further comprising incubating the biological sample in the cryopreservation composition for a predetermined incubation time between the combining and the cooling.

3. The cryopreservation process of claim 2, wherein the predetermined incubation time is in the range of two hours to eight hours.

4. The cryopreservation process of claim 1 further comprising heating the biological sample from the cryopreservation temperature after the step of cooling.

5. The cryopreservation process of claim 1, wherein the cooling occurs at a rate selected to maintain cryopreservation of the biological sample.

6. The cryopreservation process of claim 1, wherein the biological sample comprises a plurality of cells.

7. The cryopreservation process of claim 1, wherein the biological sample comprises a protein.

8. The cryopreservation process of claim 1, wherein the biological sample comprises a nucleic acid.

9. The cryopreservation process of claim 1, wherein the biological sample comprises a plasmid.

10. The cryopreservation process of claim 1, wherein the composition comprises at least one nanoparticle comprising the at least one glycerol ester component.

11. The cryopreservation process of claim 1, wherein the composition comprises at least one microparticle comprising the at least one glycerol ester component.

12. The cryopreservation process of claim 1, wherein the cryopreservation temperature is a temperature in the range of −20° C. to −200° C.

13. The cryopreservation process of claim 1, wherein the at least one glycerol ester component comprises at least one glycerol-sebacate component.

14. A cryopreservation composition comprising cell culture media containing at least one glycerol ester component.

15. The cryopreservation composition of claim 14, wherein the at least one glycerol ester component comprises at least one glycerol-sebacate component.

16. The cryopreservation composition of claim 14, wherein at least a portion of the at least one glycerol ester component has a molecular weight in the range of 200 Da to 1200 Da.

17. The cryopreservation composition of claim 14 further comprising at least one modification to the at least one glycerol ester component, wherein the at least one modification is covalently attached to the glycerol ester component and the at least one modification is selected from the group consisting of a urethane, an acrylate, an amino acid, a peptide, a fatty acid, a diacid smaller than sebacic acid, a multifunctional acid, a sugar, a cholesterol, a vitamin, or a combination thereof.

18. A cryopreserved system comprising a biological sample in a cryopreservation composition at a cryopreservation temperature, wherein the cryopreservation composition comprises at least one glycerol ester component.

19. The cryopreserved system of claim 18, wherein the at least one glycerol ester component comprises at least one glycerol-sebacate component.

20. The cryopreserved system of claim 18, wherein the cryopreservation temperature is a temperature in the range of −20° C. to −200° C.
